(19) United States
(12) Patent Application Publication
(10) Pub. No.: US 2025/0375616 A1
(43) Pub. Date: Dec. 11, 2025

(54) SIGNAL GENERATOR FOR A NEUROLOGICAL STIMULATION SYSTEM AND METHOD FOR NEUROLOGICAL STIMULATION

(71) Applicant: BIOTRONIK SE & Co. KG, Berlin (DE)

(72) Inventors: Pamela Shamsie Victoria Riahi, Portland, OR (US); Sean Slee, Tigard, OR (US); Andrew B. Kibler, Lake Oswego, OR (US)

(21) Appl. No.: 18/877,072

(22) PCT Filed: Jun. 15, 2023
(86) PCT No.: PCT/EP2023/066104

§ 371 Date: Dec. 19, 2024

Related U.S. Application Data
(60) Provisional application No. 63/356,623, filed on Jun. 29, 2022.

(30) Foreign Application Priority Data
Sep. 13, 2022 (EP) 22195423.3

(51) Int. Cl. A61N 1/36; A61N 1/05

(52) U.S. Cl. CPC A61N 1/36185; A61N 1/0551; A61N 1/36062; A61N 1/36139; A61N 1/3615; A61N 1/36171; A61N 1/36175

(57) ABSTRACT

A signal generator (100) for a neurological stimulation system includes a signal generation module (110) connectable to a plurality of implantable electrodes (11-18; 21-28) and configured to provide electrical signals to the plurality of implantable electrodes (11-18; 21-28) for neurological stimulation. The signal generator further includes controller (120) configured to operate the signal generation module (110). The controller (120) is configured to automatically change a stimulation parameter used in stimulation therapy at time intervals or at a trigger.

Embodiments of the present disclosure relate to a signal generator for a neurological stimulation system, a neurological stimulation system, a method for neurological stimulation and a machine-readable storage medium. Embodiments of the present disclosure particularly relate to a prevention of stimulation therapy habituation.

Medical implants are widely used to treat, replace, support and/or enhance biological structures of patients. For example, implantable neurological stimulation systems can be used to treat chronic diseases such as chronic pain, incontinence, Parkinson, depression, and epilepsy. Implantable neurological stimulation systems generally have an implantable signal generator and leads that supply electrical signals generated by the signal generator to neurological tissue.

Patients treated by neurological stimulation can sometimes experience therapy habituation, where their initial pain relief experienced in the first few months after implant starts to fade away. An easy workaround to this challenge is to manually reprogram the neurological stimulation system. However, the manual reprogramming requires waiting for the patient to feel pain again and notify the medical personnel to take action, which enables pain to be experienced for a certain period of time until reprogramming takes effect. In addition, the manual reprogramming requires setting up an appointment, in-office or remotely, between the patient and the medical personnel, which takes time on both side and costs money.

In light of the above, a signal generator for a neurological stimulation system, a neurological stimulation system, and a method for neurological stimulation that overcome at least some of the problems in the art are beneficial.

It is an object of the present disclosure to provide a signal generator for a neurological stimulation system, a neurological stimulation system, a method for neurological stimulation and a machine-readable storage medium which can provide an efficient control of neurological stimulation therapy. A further object of the present disclosure is to improve the long-term pain relief and/or therapy satisfaction of patients by preventing both therapy habituation and consequently pain flare up and reducing the need for undesired medical intervention.

The objects are solved by the features of the independent claims. Preferred embodiments are defined in the dependent claims.

According to an independent aspect of the present disclosure, a signal generator for a neurological stimulation system is provided. The signal generator includes a signal generation module connectable to a plurality of implantable electrodes and configured to provide electrical signals to the plurality of implantable electrodes for neurological stimulation; and a controller configured to operate the signal generation module, wherein the controller is configured to automatically change an stimulation parameter used in stimulation therapy at time intervals.

According to an embodiment, the controller is configured to automatically change an stimulation parameter used in stimulation therapy at a trigger.

According to an embodiment, the stimulation parameter is at least one of an electrode selection of the plurality of implantable electrodes, a stimulation frequency, a stimulation pulse width, or stimulation amplitude, or duty cycle.

Accordingly, an electrode selection, such as a number and/or location of electrodes, used in stimulation therapy is automatically changed by the signal generator on a regular or irregular basis. In other words, the electrode selection is different for different therapy cycles, which can achieve efficient control of neurological stimulation therapy. In addition, automatically changing the electrode selection or other therapy parameters such as amplitude, duty cycle, pulse width or stimulation frequency can reduce pain relapse due to habituation to the therapy and reduce the number and/or cost of medical interventions to prevent or resolve loss of pain relief.

The term “therapy cycle” as used throughout the present disclosure refers to a time interval during which the same electrode selection is used. When the electrode selection is changed, a new therapy cycle begins.

The signal generation module and the controller can be provided in the same hardware module, such as an implantable signal generator, or can be provided in separate hardware modules.

According to some embodiments, which can be combined with other embodiments described herein, the signal generation module can be a pulse generator. The signal generation module, such as the pulse generator, generates the electrical signals having a particular amplitude, pulse width, frequency and/or duty cycle.

According to some embodiments, which can be combined with other embodiments described herein, the controller is configured to change the stimulation parameter by selecting a different subset of stimulation parameters for stimulation therapy. In particular, for changes to the electrode selection, the controller is configured to change the electrode selection by selecting a different subset of electrodes of the plurality of implantable electrodes for stimulation therapy. For example, a first subset of electrodes can be used in a first therapy cycle and a second subset of electrodes different from the first subset of electrodes can be used in a second therapy cycle following the first therapy cycle.

Preferably, a subset of electrodes is defined by a number and/or location of the electrodes thereof. For example, the plurality of implantable electrodes can be arranged sequentially along one or more elongated leads. In one example, a first subset could include a first, third and fifth electrode of the sequence, and a second subset could include a second, fourth and sixth electrode of the sequence. In another example, a first subset could include a first number of electrodes, and a second subset could include a second number of electrodes different from the first number. In yet another example, the first subset and the second subset could be selected based on a relative location of the electrodes with respect to, for example, the patient's spinal cord or a particular region of the spinal cord.

According to some embodiments, which can be combined with other embodiments described herein, the controller is configured to change the electrode selection by shifting an electrode selection upward or downward. For example, the plurality of implantable electrodes can be arranged sequentially along one or more elongated leads. The electrode selection can be changed by shifting the electrodes upward or downward along the one or more elongated leads by a predefined number, such as one, two or three. In one example, a first subset could include a first, third and fifth electrode of the sequence, and a second subset could include a second, fourth and sixth electrode of the sequence. This corresponds to a downward shift by the predefined number of one. In another example, a first subset could include a first, third and fifth electrode of the sequence, and a second subset could include a third, fifth and seventh electrode of the sequence. This corresponds to a downward shift by the predefined number of two.

According to some embodiments, which can be combined with other embodiments described herein, the controller is configured to change the electrode selection without changing one or more signal characteristics of the electrical signals. In other words, only the electrode selection is changed, while the electrical signals delivered to the neurological tissue can remain essentially the same throughout the different therapy cycles.

According to an embodiment, the controller is configured to one or more signal characteristics of the electrical signals without changing the electrode selection. In other words, only a signal parameter of the electrical signals delivered to the neurological tissue is changed, while the electrode selection can remain essentially the same throughout the different therapy cycles.

However, the present disclosure is not limited thereto, and the controller can be configured to change the electrode selection and/or one or more stimulation parameters of the electrical signals.

Preferably, the one or more stimulation parameters are selected from the group including (or consisting of), an amplitude, a pulse width, a frequency, and a duty cycle of the electrical signals.

According to some embodiments, which can be combined with other embodiments described herein, the controller is configured to operate the signal generation module in a plurality of program modes, wherein each program mode is associated with one or more different stimulation parameters used in the stimulation therapy performed in accordance with the program mode.

The term “program mode” or “program” as used throughout the present disclosure refers to a combination of parameters used to perform the stimulation therapy for treating the patient's symptoms. A program mode may include parameters such as one or more electrode selections and one or more signal characteristics of the electrical signals (e.g., amplitude, pulse width, frequency, and/or duty cycle). However, the present disclosure is not limited thereto, and other parameters which define and/or influence the stimulation therapy can be part of the program mode(s).

According to some embodiments, which can be combined with other embodiments described herein, the controller is configured to change the electrode selection by changing from a current program mode of the plurality of program modes to another program mode of the plurality of program modes. For example, a first program mode can use a first electrode selection and/or a first set of stimulation parameters and a second program mode can use a second electrode selection and/or a second set of stimulation parameters different from the first electrode selection and/or first set of stimulation parameters. By changing from the first program mode to the second program mode or vice versa, also the electrode selection and/or the stimulation parameters are changed.

According to some embodiments, which can be combined with other embodiments described herein, the controller is configured to change the electrode selection by changing from one electrode selection of a current program mode to another electrode selection of the current program mode. In particular, a particular program mode can have two or more different electrode selections such that the electrode selection can be changed without changing the program mode.

According to some embodiments, which can be combined with other embodiments described herein, the time interval between changes of the electrode selection is a periodic interval or an aperiodic interval.

The periodic interval and/or the aperiodic interval can be within the range of one or more days or one or more weeks (e.g., two to four weeks, five weeks, six weeks, seven weeks, eight weeks).

According to an embodiment of the present invention, therapy changes are triggered by an external device or external system/server like a central networked controller (e.g. a remote service center). The controller receives patient data feeds on a regular basis, such as the patient's condition, etiology, changes in physiological function, changes in the patient's behavior, changes in the patient's pain score, previous or other patient data, wherein the controller is configured to analyze said patient data. The controller is furthermore configured to output a decision regarding the time point of therapy changes based on said patient data and/or the analyses of the patient data. The decision may be not to request a therapy change to avoid habituation at a given time.

According to an embodiment, at least one of the stimulation parameter, the time interval or the trigger is determined by the controller or by an external device or an external server. The time interval or trigger is determined based on input data on at least one of an initial programming of the signal generator, implantation parameters, a patient's condition, etiology, changes in physiological function, changes in the patient's behavior, or changes in the patient's pain score.

According to some embodiments, which can be combined with other embodiments described herein, the controller is configured to (automatically) determine the electrode selection and/or the time interval based on one or more circumstance parameters.

Preferably, the one or more circumstance parameters relate to a patient (e.g., pain reception, acute pain, etc.), an initial programming of the signal generator (e.g., an initial program mode and/or initial therapy parameters), and/or implantation parameters (e.g., lead location, time of implantation, etc.).

According to some embodiments, which can be combined with other embodiments described herein, the controller is configured to determine the electrode selection and/or the time interval (or a cycling mode which defines the electrode selection and/or the time interval) using a rule-based process and/or artificial intelligence. In particular, the cycling mode definition can be done automatically by a rule-based algorithm (e.g. an algorithm automatically generates cycling programs based on the initial program) or an artificial intelligence method (e.g. an algorithm that was trained on a large amount of patient programming data to predict the best programs that can be cycled through for a given patient based on a certain number of variable such as initial program, perception threshold, lead location, etc.).

The term “artificial intelligence” as used throughout the present application may be understood in the sense of software components or software instances which are designed to correctly interpret data, to learn from such data, and to use those learnings to provide a medical support function through flexible adaptation.

According to some embodiments, which can be combined with other embodiments described herein, the controller is configured to implement a machine learning algorithm. For example, a neural network can be implemented to determine the cycling mode, i.e., the electrode selection and the time interval between the changes.

The term “machine learning algorithm” as used throughout the present application refers to an algorithm that builds a model based on training data, in order to make predictions or decisions without being explicitly programmed to do so.

A neural network is based on a collection of connected nodes. A node that receives a signal processes the signal and can signal network neurons connected to it. Typically, nodes are aggregated into layers. Different layers may perform different transformations on their inputs. Signals travel from an input layer to an output layer. Such a neural network may be trained by processing examples, each of which contains a known input and result, forming probability-weighted associations between the two, which are stored within the data structure of the neural network. Thus, the neural network learns to perform tasks by considering given examples.

According to another independent aspect of the present disclosure, a neurological stimulation system is provided. The neurological stimulation system includes a plurality of implantable electrodes configured to deliver electrical signals to neurological tissue, such as a spinal cord; and the signal generator of the embodiments of the present disclosure.

According to some embodiments, which can be combined with other embodiments described herein, the neurological stimulation system is configured for spinal cord stimulation. However, the present disclosure is not limited thereto, and the neurological stimulation system may also be used in other stimulation therapies which use electrical signals that are delivered to electrodes implanted in a patient's body.

According to another independent aspect of the present disclosure, a method for neurological stimulation is provided. The method includes determining, by a controller, a first subset of electrodes of a plurality of implantable electrodes and performing neurological stimulation by applying electrical signals to the first subset of electrodes; and determining, by the controller, a second subset of electrodes of the plurality of implantable electrodes and performing neurological stimulation by applying electrical signals to the second subset of electrodes.

According to some embodiments, which can be combined with other embodiments described herein, determining the second subset of electrodes includes shifting an electrode selection corresponding to the first subset of electrodes upwards or downwards to obtain the second subset of electrodes.

According to some embodiments, which can be combined with other embodiments described herein, the electrical signals applied to the first subset of electrodes and the second subset of electrodes have the same signal characteristics, in particular the same amplitude and/or pulse width and/or frequency and/or duty cycle.

Embodiments are also directed at systems/devices for carrying out the disclosed methods and include system/device aspects for performing each described method aspect. These method aspects may be performed by way of hardware components, a computer programmed by appropriate software, by any combination of the two or in any other manner. Furthermore, embodiments according to the invention are also directed at methods for operating the described device/system. It includes method aspects for carrying out every function of the device/system.

According to another independent aspect of the present disclosure, a machine-readable storage medium is provided. The machine-readable storage medium includes instructions executable by one or more processors to implement the method for neurological stimulation of the embodiments of the present disclosure.

The machine-readable storage medium may include, for example, semiconductor memory devices such as Electrically Programmable Read-Only Memory (EPROM), Electrically Erasable Programmable Read-Only Memory (EEPROM), and the like. The machine-readable storage medium may be used to tangibly retain computer program instructions or code organized into one or more modules and written in any desired computer programming language. When executed by, for example, one or more processors such computer program code may implement the method for neurological stimulation of the embodiments of the present disclosure.

So that the manner in which the above recited features of the present disclosure can be understood in detail, a more particular description of the disclosure, briefly summarized above, may be had by reference to embodiments. The accompanying drawings relate to embodiments of the disclosure and are described in the following:

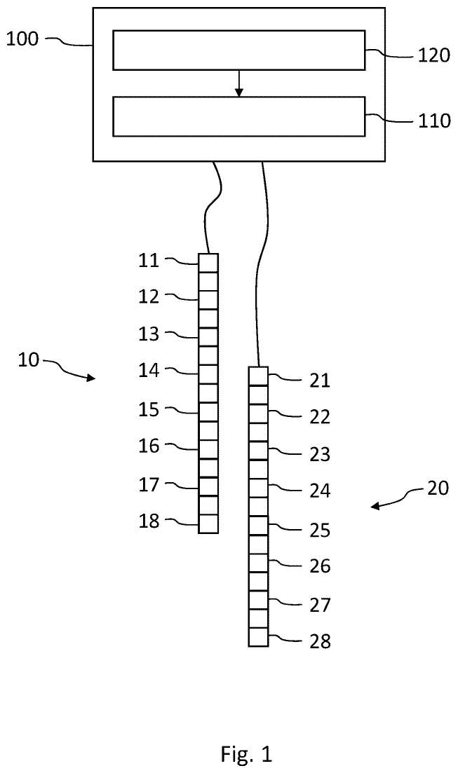

FIG. 1 shows a schematic view of a neurological stimulation system according to embodiments of the present disclosure;

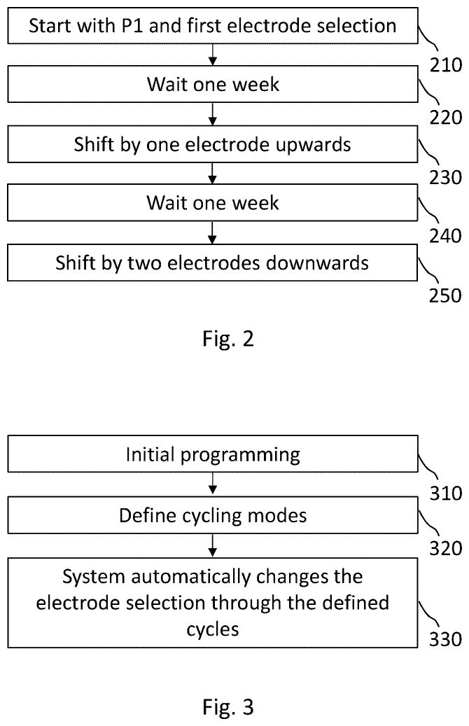

FIG. 2 shows a flowchart of an automatic change of an electrode selection according to an embodiment of the present disclosure; and

FIG. 3 shows a flowchart of an automatic change of an electrode selection according to further embodiments of the present disclosure.

Reference will now be made in detail to the various embodiments of the disclosure, one or more examples of which are illustrated in the figures. Within the following description of the drawings, the same reference numbers refer to same components. Generally, only the differences with respect to individual embodiments are described. Each example is provided by way of explanation of the disclosure and is not meant as a limitation of the disclosure. Further, features illustrated or described as part of one embodiment can be used on or in conjunction with other embodiments to yield yet a further embodiment. It is intended that the description includes such modifications and variations.

Patients treated by neurological stimulation can sometimes experience therapy habituation, where their initial pain relief experienced in the first few months after implant starts to fade away. The embodiments of the present disclosure automatically change an electrode selection over time at regular or irregular intervals, preferably every 2 to 4 weeks, to prevent therapy habituation and loss of pain relief.

FIG. 1 shows a schematic view of a neurological stimulation system according to embodiments of the present disclosure.

The neurological stimulation system includes a signal generator 100 and a plurality of implantable electrodes configured to deliver electrical signals to neurological tissue. In the example of FIG. 1, the neurological stimulation system is configured for spinal cord stimulation.

In some embodiments, the plurality of implantable electrodes, such as ring electrodes, can be arranged sequentially along one or more elongated leads. In the example of FIG. 1, a plurality of first electrodes 11-18 is arranged sequentially along a first lead 10, and a plurality of second electrodes 21-28 is arranged sequentially along a second lead 20.

The plurality of first electrodes 11-18 and/or the plurality of second electrodes 21-28 are configured for delivering therapy to the patient after implantation. In particular, the plurality of implantable electrodes, such as the plurality of first electrodes 11-18 and/or the plurality of second electrodes 21-28, can be implanted at or near a spinal cord to direct electrical signals into the patient's tissue for spinal cord stimulation.

The signal generator 100 can be implanted subcutaneously and electrically connected to the plurality of implantable electrodes. The signal generator 100 generates electrical signals to be delivered to the patient via the plurality of implantable electrodes. In particular, the signal generator 100 can be a pulse generator. The pulse generator generates the electrical signals having a particular amplitude, pulse width, frequency and/or duty cycle suitable for stimulation therapy.

In more detail, the signal generator 100 includes a signal generation module 110 connectable to the plurality of implantable electrodes and configured to provide the electrical signals to the plurality of implantable electrodes for neurological stimulation; and a controller 120 configured to operate the signal generation module 110, wherein the controller 120 is configured to automatically change an electrode selection of the plurality of implantable electrodes used in stimulation therapy at time intervals.

Accordingly, an electrode selection, such as a number and/or location of electrodes, used in stimulation therapy is automatically changed by the signal generator 100 on a regular or irregular basis. In other words, the electrode selection is different for different therapy cycles, which can achieve efficient control of neurological stimulation therapy. In addition, automatically changing the electrode selection can reduce pain relapse due to habituation to the therapy and reduce the number and/or cost of medical interventions to prevent or resolve loss of pain relief.

In a non-limiting example and by way of reference to FIG. 1, a best location for stimulation therapy can be around the electrodes 16 and 23. In a first therapy cycle, a first electrode selection can include the electrodes 14, 16 and 18. In a second therapy cycle after the first therapy cycle, a second electrode selection can include the electrodes 21, 23 and 25. In a third therapy cycle after the second therapy cycle, a third electrode selection can include the electrodes 13, 15 and 17. In a fourth therapy cycle after the third therapy cycle, a fourth electrode selection can include the electrodes 22, 24 and 26. Thereby, therapy habituation can be prevented.

According to some embodiments, the controller 120 is configured to change the electrode selection without changing one or more signal characteristics of the electrical signals, such as an amplitude, a pulse width, and/or a frequency. In other words, only the electrode selection is changed, while the electrical signals delivered to the neurological tissue can remain essentially the same throughout the different therapy cycles.

In some embodiments, the controller 120 can be configured to operate the signal generation module 110 in a plurality of program modes, wherein each program mode is associated with one or more electrode selections used in the stimulation therapy performed in accordance with this program mode. The term “program mode” or “program” as used throughout the present disclosure refers to a combination of parameters, such as one or more electrode selections and one or more signal characteristics of the electrical signals, used to perform the stimulation therapy for treating the patient's symptoms.

The controller 120 can be configured to change the electrode selection by changing from a current program mode of the plurality of program modes to another program mode of the plurality of program modes. For example, a first program mode can use a first electrode selection and a second program mode can use a second electrode selection different from the first electrode selection. Accordingly, by changing from the first program mode to the second program mode or vice versa, also the electrode selection is changed.

In one example, the therapy can start with a first program mode P1. After a certain time, such as every one or two weeks, the program mode can be switched to a next program mode (e.g., P2, . . . , Pn, P1, . . . , Pn etc.). Accordingly, the program modes can be cycled.

In another example, the therapy can start with a first program mode P1. For example, the first program mode P1 can correspond to multiphase therapy, which is a type of sub-perception therapy. After a certain time, such as every month, it can be switched to another type of sub-perception therapy (e.g., a second program mode P2 corresponding to high frequency stimulation). Then the month after, it can be switched back to the first program mode P1. Accordingly, the program modes can be cycled.

In the above examples, the program modes and thus the electrode selections are switched. In other examples, the program mode is not switched, but the controller 120 switches between different electrode selections of the same program mode.

FIG. 2 shows a flowchart of an automatic change of an electrode selection according to an embodiment of the present disclosure. FIG. 2 is an example in which the neurological stimulation system switches between different electrode selections of the same program mode.

In block 210, the therapy begins with a first program mode P1 and a first electrode selection thereof. The first electrode selection may include a second electrode (e.g., electrode 12 and/or electrode 22 in FIG. 1), a fourth electrode (e.g., electrode 14 and/or electrode 24 in FIG. 1) and a sixth electrode (e.g., electrode 16 and/or electrode 26 in FIG. 1).

After one week (block 220), the neurological stimulation system selects in block 230 a second electrode selection by shifting the electrodes by one electrode upwards. The second electrode selection then includes a first electrode (e.g., electrode 11 and/or electrode 21 in FIG. 1), a third electrode (e.g., electrode 13 and/or electrode 23 in FIG. 1) and a fifth electrode (e.g., electrode 15 and/or electrode 25 in FIG. 1).

After another week (block 240), the neurological stimulation system selects in block 250 a third electrode selection by shifting the electrodes by two electrodes downwards. The third electrode selection then includes the third electrode (e.g., electrode 13 and/or electrode 23 in FIG. 1), the fifth electrode (e.g., electrode 15 and/or electrode 25 in FIG. 1) and a seventh electrode (e.g., electrode 17 and/or electrode 27 in FIG. 1).

After another week, the neurological stimulation system can select the first electrode selection again. Accordingly, the electrode selections can be cycled.

FIG. 3 shows a flowchart of an automatic change of an electrode selection according to further embodiments of the present disclosure.

In an initial programming session (block 310), a range of program modes or programs can be defined within the signal generator, wherein each program mode or program has one or more electrode selections. The initial programming session can be done during or shortly after implantation.

During the same programming session or e.g. after a week of baseline operation, cycling modes for cycling of the electrode selection can be defined. For example, the cycling mode definition can be done automatically by a rule-based algorithm (e.g. an algorithm automatically generates cycling programs based on the initial program) or an artificial intelligence method (e.g. an algorithm that was trained on a large amount of patient programming data to predict the best programs that can be cycled through for a given patient based on a certain number of variable such as initial program, perception threshold, lead location, etc.). For example, a recommender system is a category of machine learning algorithm that can provide suggestions of cycling modes and/or cycling programs. A content-based model is a subset of recommender systems that takes into account the current patient's program settings and a data pool of successful program cycling with previous patients in order to produce relevant program cycling suggestions. According to an embodiment, collaborative filtering is another subset of ML algorithm that can provide program cycling suggestions based on similarities between the characteristics and programs of a current patient and those of previous patients, of which program cycling outcomes are already known and can be leveraged to make suggestions for the current patient.

In block 330, the stimulator automatically changes the electrode selection e.g. by changing programs through the defined cycles.

Accordingly, an electrode selection is automatically changed on a regular or irregular basis. Automatically changing the electrode selection can reduce pain relapse due to habituation to the therapy and reduce the number and/or cost of medical interventions to prevent or resolve loss of pain relief.

While the foregoing is directed to embodiments of the disclosure, other and further embodiments of the disclosure may be devised without departing from the basic scope thereof, and the scope thereof is determined by the claims that follow.

1. A signal generator (100) for a neurological stimulation system, comprising:
a signal generation module (110) connectable to a plurality of implantable electrodes (11-18; 21-28) and configured to provide electrical signals to the plurality of implantable electrodes (11-18; 21-28) for neurological stimulation; and
a controller (120) configured to operate the signal generation module (110), wherein the controller (120) is configured to automatically change a stimulation parameter, used in stimulation therapy at time intervals.

2. The signal generator (100) of claim 1, wherein the stimulation parameter is at least one of an electrode selection of the plurality of implantable electrodes (11-18; 21-28), stimulation frequency, stimulation pulse width, stimulation amplitude, or duty cycle.

3. The signal generator (100) of claim 2, wherein the controller (120) is configured to change the electrode selection by selecting a different subset of electrodes of the plurality of implantable electrodes (11-18; 21-28) for stimulation therapy.

4. The signal generator (100) of claim 2, wherein the controller (120) is configured to change the electrode selection by shifting an electrode selection upward or downward.

5. The signal generator (100) of claim 1, wherein the controller (120) is configured to change the stimulation parameter by changing an electrode selection without changing one or more signal characteristics of the electrical signals, wherein the one or more signal characteristics are selected from the group consisting of an amplitude, a pulse width, a frequency, and a duty cycle.

6. The signal generator (100) of claim 1, wherein the controller (120) is configured to operate the signal generation module (110) in a plurality of program modes, wherein each program mode is associated with one or more stimulation parameters used in the stimulation therapy performed in accordance with the program mode.

7. The signal generator (100) of claim 1, wherein the time interval is a periodic interval or an aperiodic interval, within the range of one or more days or one or more weeks.

8. The signal generator (100) of claim 1, wherein at least one of the stimulation parameter, the time interval, or the trigger is determined by the controller (120) or by an external device or an external server,
wherein the time interval or trigger is determined based on input data on at least one of an initial programming of the signal generator, implantation parameters, a patient's condition, etiology, changes in physiological function, changes in the patient's behavior, or changes in the patient's pain score.

9. The signal generator (100) of claim 1, wherein the controller (120) is configured to determine one or both of the stimulation parameter or the time interval using a rule-based process and/or artificial intelligence.

10. A neurological stimulation system, comprising:
a plurality of implantable electrodes (11-18; 21-28) configured to deliver electrical signals to neurological tissue; and
the signal generator (100) of claim 1.

11. The neurological stimulation system of claim 10, wherein the neurological stimulation system is configured for spinal cord stimulation.

12. A method for neurological stimulation, comprising:
determining, by a controller (120), a first subset of electrodes of a plurality of implantable electrodes (11-18; 21-28) and performing neurological stimulation by applying electrical signals to the first subset of electrodes; and
determining, by the controller (120), a second subset of electrodes of the plurality of implantable electrodes (11-18; 21-28) and performing neurological stimulation by applying electrical signals to the second subset of electrodes.

13. The method of claim 12, wherein determining the second subset of electrodes includes shifting an electrode selection corresponding to the first subset of electrodes upwards or downwards to obtain the second subset of electrodes.

14. The method of claim 12- or 13, wherein the electrical signals applied to the first subset of electrodes and the second subset of electrodes have the same signal characteristics, including one or more of the same amplitude, the same pulse width, the same frequency, or the same duty cycle.

15. A machine-readable storage medium, comprising instructions executable by one or more processors to implement the method for neurological stimulation of claim 12.

16. The signal generator (100) of claim 1, wherein the controller (120) is configured to change the stimulation parameter by changing one or more signal characteristics of the electrical signals, wherein the one or more signal characteristics are selected from the group consisting of an amplitude, a pulse width, a frequency, and a duty cycle, without changing an electrode selection.
